(19) United States
(12) Patent Application Publication
(10) Pub. No.: US 2015/0112238 A1
(43) Pub. Date: Apr. 23, 2015

(54) NECK BRACES

(71) Applicant: HANDSOME LIMITED, Radcliffe (GB)

(72) Inventors: Matthew Cockerill, London (GB); Jamie Cobb, London (GB); John Sutcliffe, London (GB)

(73) Assignee: HANDSOME LIMITED, Radcliffe (GB)

(21) Appl. No.: 14/354,672

(22) PCT Filed: Oct. 29, 2012
(86) PCT No.: PCT/GB2012/052690

§ 371 Date: Dec. 1, 2014

(30) Foreign Application Priority Data
Oct. 27, 2011 (GB) 1118604.6

(51) Int. Cl. A61N 1/36; A61F 5/30; A61F 5/055

(52) U.S. Cl. CPC A61N 1/36003; A61F 5/055; A61F 5/30
USPC 602/18; 602/2

(57) ABSTRACT

A neck brace comprises a frame, which includes a support section for supporting a user's lower jaw and a brace section for bracing the support section. The brace section comprises a sternum rest and a strut that joins the sternum rest to the remainder of the frame, and is shaped to avoid contact with the user's clavicle region.

The present invention relates to neck braces for use in the alleviation of medical conditions of the neck.

Neck braces, such as cervical collars, are well known for supporting the neck of a user who suffers from damage to or around the neck area, caused by injury or disease. Users suffering from such damage often find that holding the head in an upright position is painful and tiring. It may also be necessary to hold the head still to aid the healing process. A neck brace serves to support the head, to immobilise the head relative to the shoulders, and to minimise the pressure on the damaged area.

Conventional neck braces comprise full cervical collars that completely encircle the neck. Such neck braces are bulky, conspicuous and uncomfortable to wear. They are frequently provided with internal padding. This can relieve some discomfort by providing a cushioning effect, but heat is trapped in the padding. The result is a neck brace that is hot and unpleasant to wear, particularly in summer months.

Immobilisation of the head may be desirable, for example if minimal movement will aid recovery, or if movement of the head is painful. However, in many cases a total restriction of movement is unnecessary, and is uncomfortable and inconvenient for the user. For example, in immobilising the head, conventional neck braces also restrict downward movement of the jaw and hence hinder eating and speaking.

The conspicuous, restrictive and uncomfortable nature of a conventional neck brace discourages users from wearing the neck brace, which is entirely counterproductive. Failing to wear the neck brace, or not wearing it for the full number of prescribed hours per day, can be detrimental to the success of the treatment and can increase recovery time.

In an attempt to overcome these problems, vented neck braces have been introduced in which ventilation holes are provided in the neck brace. In practice, the improvement in comfort is minimal, and providing ventilation holes does not address the problem of bulk and obtrusiveness.

An alternative to conventional or vented neck braces is a wire frame neck brace. This comprises a frame with an upper support section that supports the jaw and a lower brace section that rests on the user's lower neck, shoulders or sternum. Wire frame neck braces that consist of wire alone, or that have only thin padding on the wire, exert an uncomfortable pressure where they contact the user's jaw, neck, shoulders or sternum. In practice, the wire must be wrapped in thick padding to relieve this pressure. As a result, such wire frame neck braces are still conspicuous, and offer little improvement over conventional neck braces.

The need for a neck brace that meets the conflicting requirements of being both comfortable for the user and inconspicuous to wear has not, therefore, been met.

The support provided by a neck brace as described above aids long-term healing of the affected area and may relieve pain to some extent by virtue of the support it gives to the user's head. However the user may still suffer ongoing pain from the underlying neck condition, which can contribute to the user's reluctance to wear a neck brace. It is a particular problem that wearing the neck brace may hinder efforts to treat the ongoing pain, for example by covering or otherwise blocking access to the location where the pain is felt.

US 2009/0264802 to Chen discloses a neck brace designed to support the head of a user following surgery on the cervical vertebrae. This is an example of a wire frame neck brace that is bulky and obtrusive due to thick padding on the wire. It also illustrates the problem of how to treat the user's neck when the neck brace is in place. The solution in Chen's case is partially to disassemble the neck brace around the back of the user's neck to provide access to a surgical wound.

Partial disassembly of a neck brace as contemplated by Chen is an unwelcome and awkward additional operation; it also leaves the bulk of the neck brace in place, where it may continue to hinder efforts to treat the ongoing pain of a neck condition.

Accordingly, it would be desirable to provide a neck brace that addresses the problems described above.

The invention may be expressed as a neck brace comprising a frame, the frame including a support section for supporting a user's lower jaw and a brace section for bracing the support section, wherein the brace section comprises a sternum rest and a strut that joins the sternum rest to the remainder of the frame and is shaped to avoid contact with the user's clavicle region.

The strut may be a bridge member shaped to provide clearance over the user's clavicle region. In one embodiment, the bridge member is curved about an axis of curvature that, in use, is substantially parallel to a line extending across the user's clavicles.

The strut may taper from the remainder of the frame toward the sternum rest and may comprise downwardly-converging arms. The strut may be V-shaped when viewed from the user's front in use, with the sternum rest being located at a downward apex of the V. An apex region of the strut may be U-shaped, and may be defined by a pair of substantially parallel side portions spaced apart by at least one cross-member.

In one embodiment, the sternum rest comprises a pad removably attachable to the strut. At least part of the strut is formed from an elongate wire, and the sternum pad has a peripheral formation shaped to engage the wire. The peripheral formation may be a groove extending at least partially around the sternum pad. The wire may exert resilient engaging force on the sternum pad.

To aid comfort, the sternum pad may have a relatively soft covering, which may be removable. The sternum pad may also comprise a TENS unit.

The support section may be formed from an elongate wire. The brace section and the support section may be formed from a continuous elongate wire. To increase comfort for the user, the support section may have a jaw rest with a flattened, elongate cross-section in a plane orthogonal to the wire of the support section.

The support section and the brace section may be spaced from each other and connected by side members.

The neck brace may also comprise a retainer for extending around the back of a user's neck to hold the support section and the brace section in a use position. The retainer may comprise a TENS electrode.

The invention may also be expressed as a neck brace comprising at least one TENS electrode and having a holder for a TENS unit for providing pulses to the TENS electrode.

The TENS unit may be removable from the holder and disconnectable from the TENS electrode.

The neck brace may comprise a support section for supporting a user's lower jaw, a brace section for bracing the support section and a retainer for extending around the back of a user's neck to hold the support section and the brace section in a use position. The TENS electrode may be on the retainer and the holder for the TENS unit may be on the brace section.

In one embodiment, the brace section is electrically conducting and the TENS electrode and a TENS unit in the holder are electrically connected via the brace section. The brace section may define a gap such that the TENS unit, when located in the holder and activated, bridges the gap to complete an electrical circuit including the TENS electrode.

The brace section may comprise a strut that extends downwardly to the holder for the TENS unit, positioned to lie adjacent the sternum of a user. The strut may comprise a pair of arms that define the holder between them. If the brace section defines a gap, the arms may be spaced apart to define that gap.

The invention extends to, in combination, a neck brace comprising at least one TENS electrode and having a holder for a TENS unit for providing pulses to the TENS electrode, and a TENS unit cooperable with or fitted to the holder.

The invention also extends to a TENS unit or a sternum pad for a neck brace, comprising a peripheral formation shaped to engage the neck brace to attach the TENS unit or the sternum pad to the neck brace.

In order that the invention may be more readily understood, reference will now be made, by way of example, to the accompanying drawings, in which:

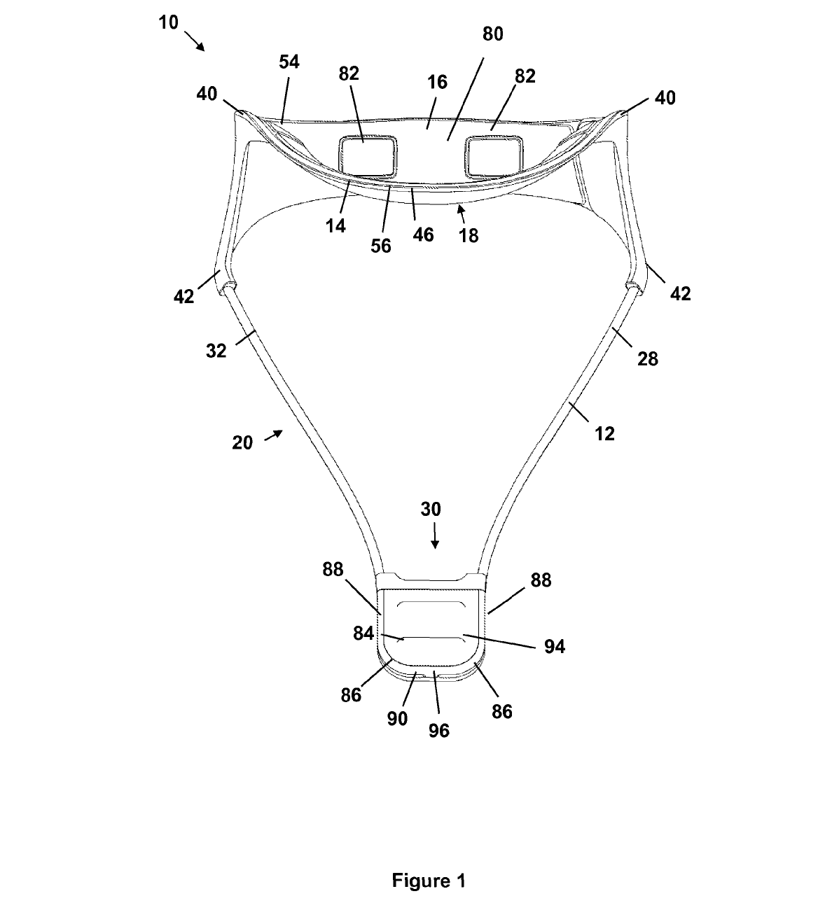

FIG. 1 is a front view of a neck brace according to the present invention;

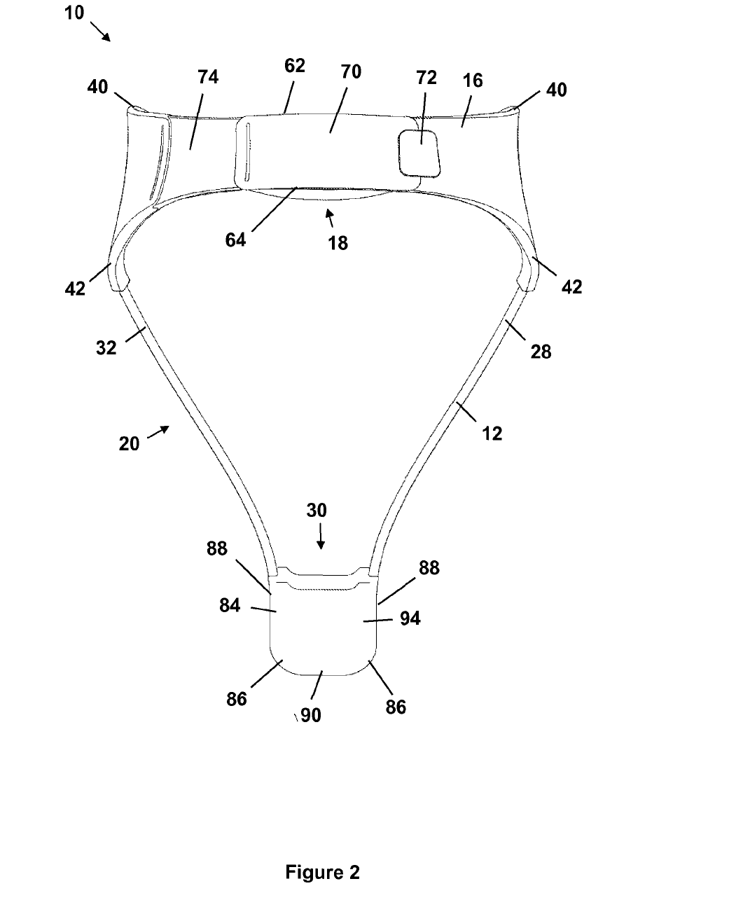

FIG. 2 is a rear view of the neck brace of FIG. 1;

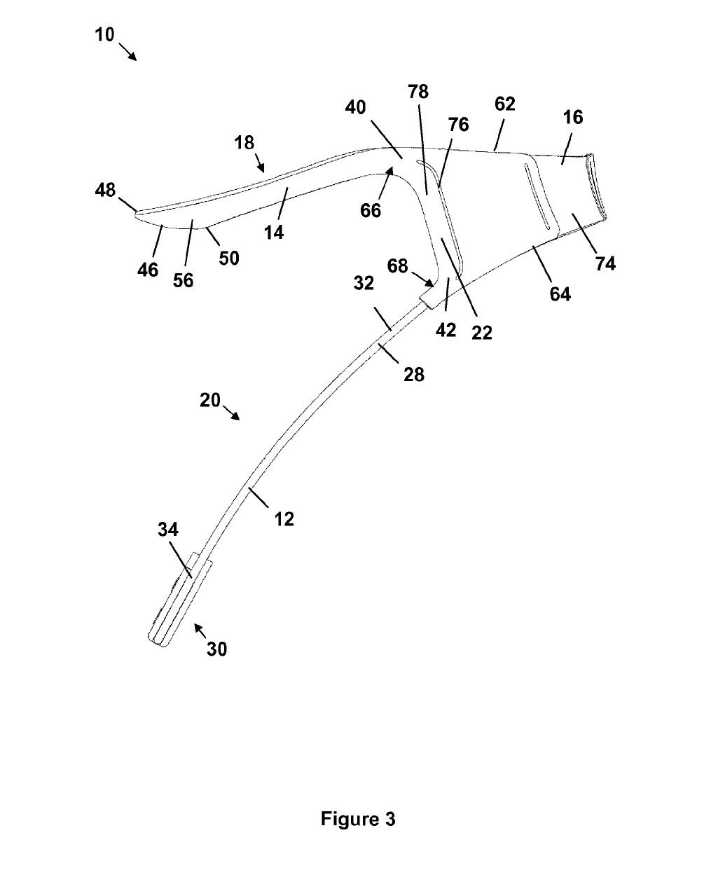

FIG. 3 is a side view of the neck brace of FIGS. 1 and 2;

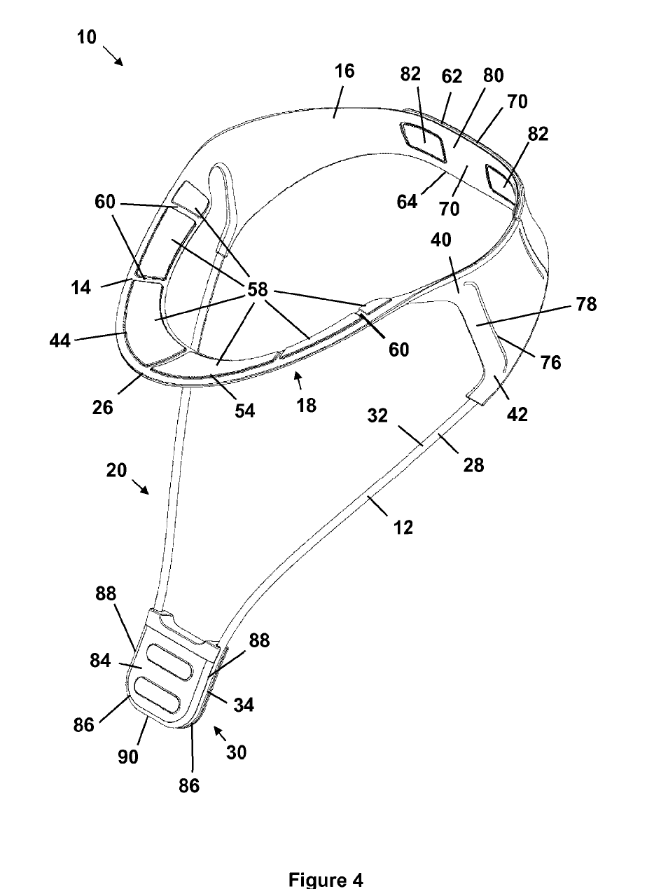

FIG. 4 is a perspective view from front, above and left side of the neck brace of FIGS. 1 to 3;

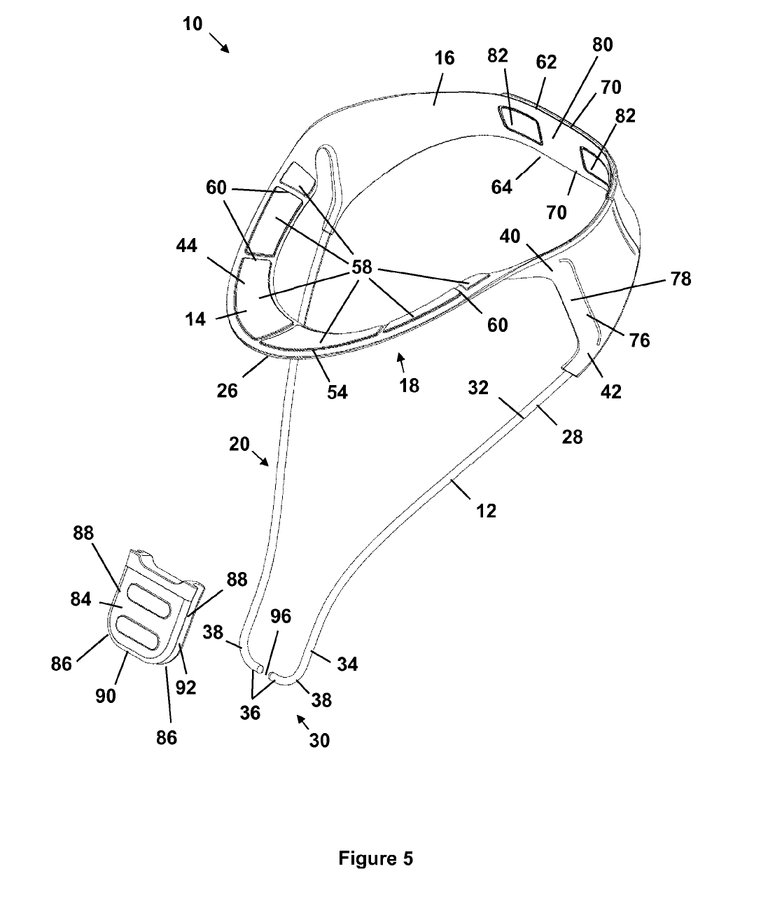

FIG. 5 corresponds to FIG. 4 but shows an optional TENS unit removed from the neck brace;

FIG. 6 is an enlarged detail perspective view of the neck brace of FIGS. 1 to 5, showing the construction of a neck strap of the neck brace;

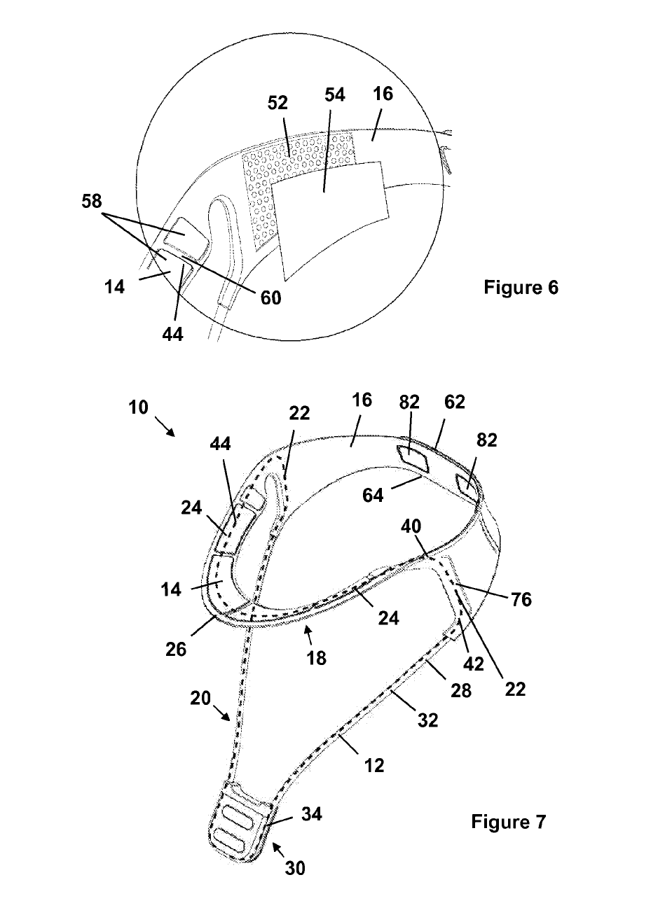

FIG. 7 is a perspective view from front, above and left side of the neck brace of FIGS. 1 to 3, showing the position on the frame within the neck strap and a jaw support;

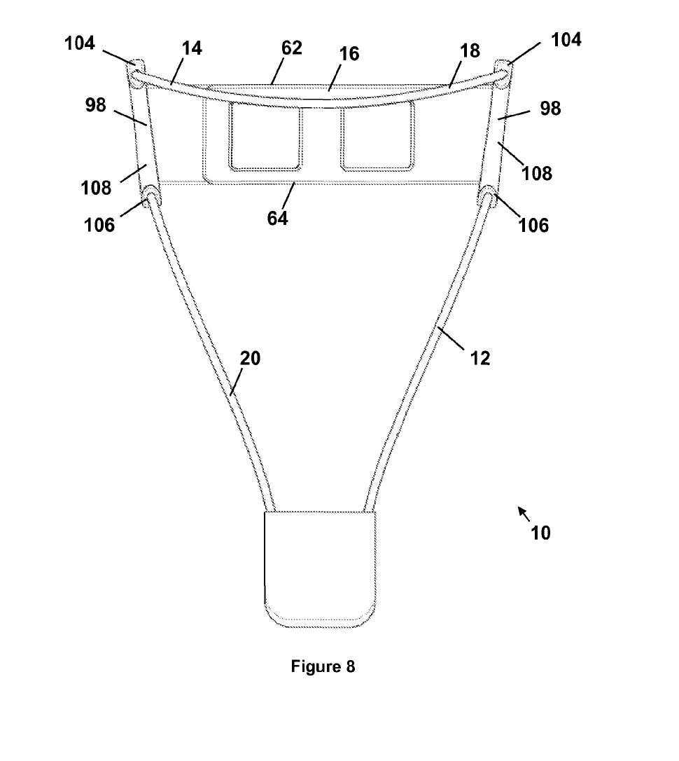

FIG. 8 is a front view of a neck brace according to an alternative embodiment of the present invention;

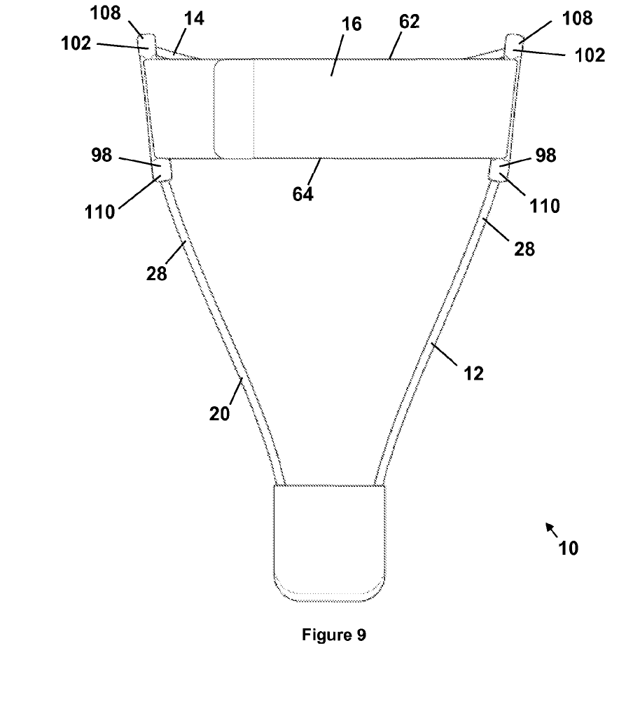

FIG. 9 is a rear view of the neck brace of FIG. 8;

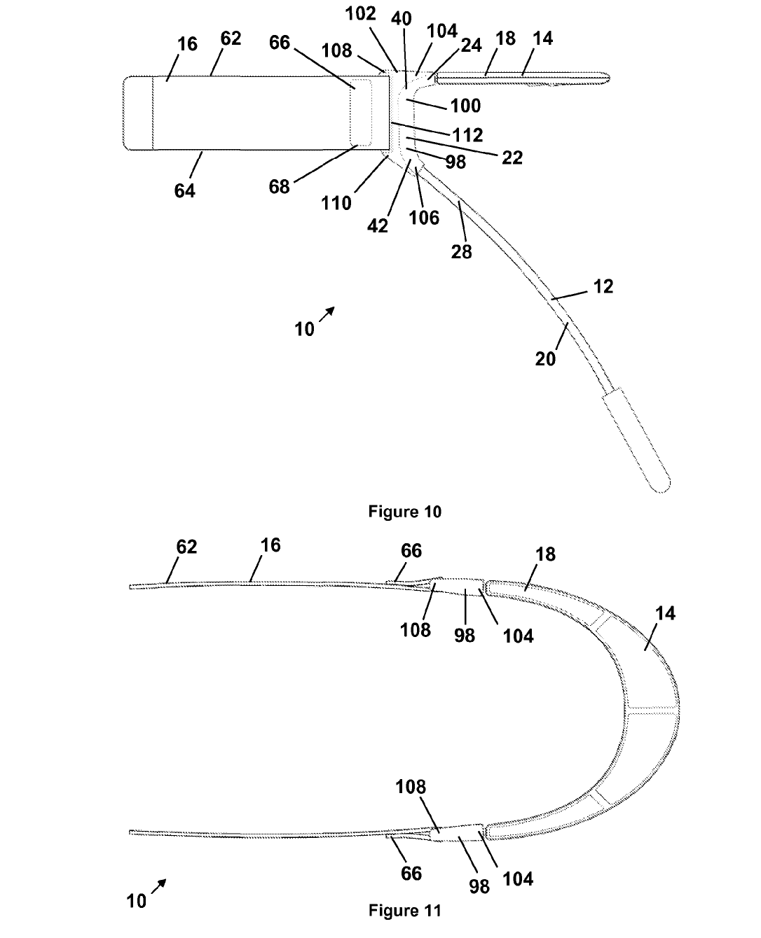

FIG. 10 is a side view of the neck brace of FIGS. 8 and 9; and

FIG. 11 is a top view of the neck brace of FIGS. 8 to 10.

Referring to FIGS. 1 to 3 of the drawings, a neck brace 10 according to the present invention comprises a frame 12 that supports a jaw rest 14. The frame 12 and the jaw rest 14 are connected to a neck strap 16 whereby the neck brace 10 is secured around a user's neck.

The frame 12 comprises an upper support section 18 to support a user's jaw and a lower brace section 20 to bear on the user's chest region. The support 18 and brace 20 sections of the frame 12 are spaced from each other and connected by generally upright side members 22 (see FIG. 3) at the rear of the frame 12, with the support 18 and brace 20 sections extending forwardly from the side members 22.

The frame 12 is formed from one or more thin elongate members, so that the neck brace is of minimal size and is as inconspicuous as possible when worn. An elongate member of the frame 12 may be made from any suitable material, such as metal or plastics. Plastics may be moulded in a desired shape. However, in the embodiment shown, the frame 12 is formed from one continuous elongate metal wire which is bent to the desired shape. The wire is flexible to allow the frame 12 to be shaped as desired; the wire is also resilient, to provide the reactive forces necessary to allow the neck brace 10 to support the user's head.

As best seen in FIG. 7, the upper, support section 18 of the frame 12 is curved with a generally parabolic shape, with side arms 24 that converge forwardly from the side members 22 to a rounded apex 26 at the forward extremity of the support section 18. The support section 18 is near-planar, with a shallow upward curve out of that plane approaching its forward end. Also, the side arms 24 of the support section 18 curve down at their rear where they extend into the respective side members 22.

When the neck brace is oriented for use, the support section 18 has an overall downward tilt from its rear to its front, such that its forward end lies below the upper extremity of its side arms 24. The shape of the support section 18 is such that when the neck brace is worn, the support section 18 follows the curve of the user's lower jaw or mandible, extending under, along and supporting the user's lower jaw including the chin.

The lower, brace section 20 comprises downwardly-converging side arms 28 that lend a general V-shape to the brace section 20 in front or rear view as shown in FIGS. 1 and 2 of the drawings. An apex region 30 of the brace section 20 near the apex of the V is generally U-shaped. More specifically, the rate of convergence of the side arms 28 decreases downwardly in a concave-curved transition between major portions 32 of the side arms 28 that converge with each other and minor portions 34 of the side arms 28 that lie substantially parallel to each other.

As best seen in FIG. 5, the minor portions 34 of the side arms 28 are spaced by inwardly-facing, opposed cross-members 36 at the downward extremity of the brace section 20, with the cross-members 36 being generally orthogonal to the minor portions 34. The corners 38 where the cross-members 36 join the minor portions 34 are radiused to prevent uncomfortable pressure on the user and to minimise stress in the frame 12 in use.

In side view as shown in FIG. 3 of the drawings, the brace section 20 is tilted steeply downwardly and forwardly to diverge forwardly from the upper support section 18. The major portions 32 of the side arms 28 of the brace section 20 are shallowly curved in side view such that their inclination to the horizontal increases moving downwardly. The minor portions 34 at the downward ends of the side arms 28 are generally straight in side view such that the U-shaped apex region 30 is generally planar.

Each side arm 28 of the brace section 20 curves up at its rear where it extends into a respective side member 22. The side members 22 are inclined forwardly and upwardly from there to the rear of the upper support section 18. The side members 22 also converge upwardly when viewed from the front or rear as shown in FIGS. 1 and 2 of the drawings. Consequently, the junctions 40 between the support section 18 and the side members 22 are above, inward and forward of the lower junctions 42 between the side members 22 and the brace section 20.

The jaw rest 14 is connected to, and supported by, the support section 18, so that it follows the curve defined by the support section 18. The jaw rest 14 has a flattened, elongate cross-section in a plane orthogonal to the wire 12 of the support section 18, with a width substantially greater than its thickness (best shown in FIG. 1). The longitudinal axis of that cross-section is inclined downwardly and inwardly around the support section 18 such that the jaw rest 14 is generally part frusto-conical or dished.

In effect, the dished shape of the jaw rest 14 defines a receptacle for the user's head, allowing the user's lower jaw to settle in a central forward-facing position. This resists pivotal movement of the user's head with respect to the shoulders. However, as the frame 12 of the neck brace 10 is preferably resilient, it is possible for the support section 18 to pivot with respect to the side members 22 and the brace section 20 to allow the user to move his or her jaw for the purposes of talking and eating.

As best shown in FIG. 4, the cross-section of the jaw rest 14 also has a shallow curve, so that the jaw rest 14 has a concave inner surface 44 and a convex outer surface 46. This curve allows the user's lower jaw to fit more comfortably into the jaw rest 14.

In the embodiment shown, the wire 12 of the support section 18 is covered by or embedded in the jaw rest 14. More specifically, the wire 12 is sandwiched between inner and outer layers of the jaw rest 14. Consequently, when the neck brace 10 is worn, the wire 12 of the support section 18 is not in contact with the user's skin.

The jaw rest 14 extends both outwardly and inwardly (viewed from the side, forwardly and rearwardly) from the wire 12 of the support section 18. In this way an outer edge 48 of the jaw rest 14 is disposed outwardly and upwardly with respect to the wire 12, and an inner edge 50 of the jaw rest 14 is disposed inwardly and downwardly with respect to the wire 12.

As a result of its construction, the jaw rest 14 is low-profile and unobtrusive, whilst providing a large surface area on which the user's lower jaw rests. This minimises pressure exerted on the user's neck and lower jaw and maximises comfort. This combination of effectiveness, comfort and sleekness encourages the user to wear the neck brace 10 of the invention, which of course is a key element of its utility.

Preferably, as shown, the jaw rest 14 is formed from a seamless, breathable material to reduce irritation caused by discontinuities in the upper surface of the jaw rest 14, and to improve transmission of heat away from the user's neck and lower jaw. The breathable material comprises a perforated foam inner core 52, as described and best shown in FIG. 6, through which air can pass. The upper or inner surface 44 of the jaw rest 14 is covered by an inner, hypoallergenic wicking fabric 54 and the outer or lower surface 46 of the jaw rest 14 is covered by an outer air-flow fabric 56. The inner 54 and outer 56 fabrics are woven or perforated to allow passage of air to increase comfort for the user. The inner 54 and outer 56 fabrics may be different, for example a relatively smooth, soft inner fabric 54 for comfort and a relatively tough outer fabric 56 for robustness.

Optionally, as shown in FIG. 4, the jaw rest 14 comprises padded regions 58 to increase comfort in areas of contact between the user and the jaw rest 14. These padded regions 58 suitably comprise thickened regions of the foam inner core 52 as shown. The padded regions 58 are suitably discrete rather than continuous to define gaps 60 between them to the further benefit of airflow.

The flexible neck strap 16 is a generally semicircular collar section that curves rearwardly between the side members 22 of the frame 12. When the neck brace 10 is worn, the neck strap 16 extends around the back of the user's neck to prevent forward movement of the neck brace 10, holding the support section 18 and the jaw rest 14 snugly under the user's lower jaw.

In side view, shown in FIG. 3, the upper 62 and lower 64 edges of the neck strap 16 converge rearwardly such that the height of the neck strap 16 decreases moving from its front to its back. More specifically, the upper edge 62 of the neck strap 16 is near-horizontal while the lower edge 64 of the neck strap 16 is inclined upwardly moving rearwardly, with a shallow curve being a continuation of the side-on curve of the brace section 20.

As shown in FIG. 2, the upper side regions 66 at the front of the neck strap extend smoothly into the jaw rest 14. The lower side regions 68 at the front of the neck strap extend downward around the junctions 42 between the brace section 20 and the side members 22, so that the neck strap 16 extends a short way down the side arms 28 of the brace section 20.

The back of the neck strap 16 comprises closure sections 70 that overlap when the neck brace 10 is worn to secure the neck brace 10 around the user's neck. The closure sections 70 are removably attachable to one another to allow the neck brace 10 to be placed around the user's neck, secured in position, and removed as required. In the embodiment shown the closure sections 70 are removably attachable by cooperating hook-and-loop fastener strips 72 disposed on an outer surface of an inner closure section 70 and an inner surface of an outer closure section 70.

The hook-and-loop fastener strips 72 allow the length of the neck strap 16 to be adjusted to fit the user's neck closely and comfortably. The neck strap 16 shown in the illustrated embodiment also comprises an elasticated region 74 to improve the fit of the neck strap 16, and to accommodate movement of the neck when the closure sections 70 are attached.

The neck strap 16 and the jaw rest 14 are formed of one piece by thermoforming, with no seams or other discontinuities that would cause discomfort, and are attached to the frame 12 by high-frequency welding. For example, the side regions 66, 68 at the front of the neck strap 16 each have a weld line 76 along which inner 54 and outer 56 layers of the neck strap 16 are welded together to form an elongate retaining pocket 78 for the side members 22 of the frame 12. This locks the frame 12 relative to the neck strap 16 and the jaw rest 14. High-frequency welding also attaches the outer 56 and inner 54 layers of the neck strap 16 and the jaw rest 14 to each other along peripheral seams, which advantageously are not in contact with the user's skin.

As shown in the detail view of FIG. 6, the neck strap 16 is made of the same breathable material as the jaw rest 14, with a perforated foam inner core 52 between inner 54 and outer 56 fabric layers.

In the preferred embodiment shown in FIG. 5 of the drawings, the inner side 80 of the neck strap 16 supports two transcutaneous electrical nerve stimulation (TENS) electrodes 82. The TENS electrodes 82 are each generally rectangular and are symmetrically positioned in spaced locations about the central point of the neck strap 16. When the neck brace 10 is in use, and the neck strap 16 is correctly adjusted, the TENS electrodes 82 are held in contact with the skin of the user's neck so that TENS pulses can be delivered directly to the user's neck for pain relief.

The TENS electrodes 82 are electrically connected to the wire frame 12. The wire frame 12 is electrically conducting, and connected to a TENS unit 84 removably attached to the U-shaped apex portion 30 of the brace section 20 (see FIG. 5). Preferably, the wire frame 12 is insulated stainless steel. The TENS unit 84 is essentially rectangular in shape with radiused lower corners 86 to match the U-shape of the apex portion 30, and has a front-to-rear thickness substantially less than its width and its height, so that it is unobtrusive, and may easily be hidden under clothing.

Side 88 and bottom 90 edges of the TENS unit 84 comprise a continuous peripheral groove 92 of semi-circular cross section that is of substantially the same radius of cross-sectional curvature as the elongate wire 12 of the brace section 20. The base-to-base distance between the grooves 92 of the side edges 88 of the TENS unit 84 is substantially equal to the internal width of the U-shaped apex region 30 of the brace section 20. In this way, the TENS unit 84 fits tightly into the apex region 30 with the wire 12 of the brace section 20 fitting into the peripheral groove 92 of the TENS unit 84.

As previously discussed, the wire 12 of the brace section 20 is flexible and resilient, which allows for elastic deformation so that the TENS unit 84 can be snap-fitted into the U-shaped apex region 30. The resilience of the wire 12 then holds the TENS unit 84 in place.

When the neck brace 10 is worn and the TENS unit 84 is inserted into the apex region 30 of the brace section 20, the underside of the TENS unit 84 rests on the user's sternum. The TENS unit 84 therefore spreads the force exerted by the brace section 20 over a larger area to reduce the pressure on the sternum. To increase the user's comfort, the underside of the TENS unit 84 may be provided with a soft covering, such as a soft rubber case 94, shown in FIGS. 1 and 2, which may be replaceable.

Advantageously, as shown, the curvature of the side arms 28 of the brace section 20 in side view holds the side arms 28 away from the user's upper chest so that they do not contact the user's collarbones or clavicles. In this way, the side arms 28 bridge the user's collarbones or clavicles so that forces are exerted on the user's sternum by the TENS unit 84 and on the users lower neck or shoulders by the upper most regions of the side arms 28.

It is beneficial that the neck brace 10 does not exert pressure on a clavicle as this is a particularly sensitive area. By bending the wire 12 of the brace section 20, the length and curvature of the side arms 28 can be tailored to the individual user if desired, for maximum comfort. However, the curvature of the side arms 28 is such that the neck brace 10 of the invention can comfortably fit a wide range of users even without individual fitting, adaptation or tailoring.

In the embodiment shown, the wire 12 of the brace section 20 is formed from an electrically-conducting material, and is part of a circuit between the TENS unit 84 and the TENS electrodes 82.

The cross-members 36 of the apex portion 30 of the brace section 20 do not touch but instead define a gap 96 in the wire frame 12. The TENS unit 84 comprises electrical terminals (not shown) positioned to connect to respective cross-members 36 and hence to bridge the gap 96 in the wire frame 12 when the TENS unit 84 is inserted into the apex portion 30. Thus, the TENS unit 84 completes the circuit to allow electrical connection between the TENS unit 84 and the TENS electrodes 82. Conversely, when the TENS unit 84 is not inserted into the brace section 20, the electrical circuit is incomplete and the TENS electrodes 82 are inoperative.

When the TENS unit 84 is inserted into the apex portion 30 and the electrical connection is complete, the user can use the TENS unit 84 to control pain-relieving pulses delivered by the TENS electrodes 82. For example, the TENS unit 84 may be used to turn the TENS function on or off, or to control the frequency and/or intensity of the TENS pulses. The TENS unit 84 therefore contains circuitry to generate and to control the TENS pulses; it also houses batteries to supply power.

When TENS functionality is not required, the TENS unit 84 can be removed. If required, it can be replaced by a dummy counterpart of the TENS unit 84 which lacks TENS functionality but completes the neck brace 10 and helps to reduce pressure on the sternum.

The integrated TENS functionality of the neck brace 10 of the invention provides relief for ongoing pain of the underlying neck condition. In addition to the advantages this provides for the user in terms of pain-relief during use, this also encourages the user to wear the neck brace 10, aiding long-term treatment of the underlying medical condition.

FIGS. 8 to 11 show an alternative embodiment of the invention. As best shown in FIG. 10, in this embodiment, the neck strap 16 and the jaw rest 14 are not formed from a continuous piece, but are instead formed from separate pieces that are separately attached to the frame 12. In this embodiment, the side members 22 are fully surrounded by covers 98 (see FIGS. 10 and 11), which are formed separately from the neck strap 16 and the jaw rest 14.

The side cover 98 fully surrounds the side member 22 to provide a cushioning effect, and to provide additional structural support. Each side cover 98 comprises a front cover portion 100 that extends around and forwardly of its respective side member 22, and a rear cover portion 102, that extends rearwardly of its respective side member 22.

The front cover portion 100 comprises an upper front cover portion 104 that extends forwardly around the junction 40 between the side member 22 and the support section 18, so that the side cover 98 extends a short distance along the side arms 24 of the support. The front cover portion 100 also comprises a lower front cover portion 106 that extends downwardly around the junctions 42 between the brace section 20 and the side members 22, so that the side cover 98 extends a short distance along the side arms 28 of the brace section 20.

The rear cover portion 102 comprises an upper rear cover portion 108 that is contiguous with the upper front cover portion 104, and a lower rear cover portion 110 that is contiguous with the lower front cover portion 106. A slit 112 is provided rearward of and substantially parallel to the side member 22, and penetrates through the thickness of the side cover 98 to provide an elongate aperture. In use, the slit 112 receives the neck strap 16.

In side view, shown in FIG. 10, the upper 62 and lower 64 edges of the neck strap 16 are parallel such that the height of the neck strap 16 remains substantially constant moving from its front to its back. The forward ends of the neck strap 16 meets the side covers 98. Specifically, the side regions 66, 68 at the forward ends of the neck strap 16 extend into the slits 112 of the side covers 98, so as to join the neck strap 16 to the frame 12 of the neck brace 10.

Optionally, and as best shown in FIG. 11, the side regions 66, 68 may extend through the slits 112 from an inner surface 114 of the side covers 98 to an outer surface 116 of the side covers 98, and may be fixed to an outer surface 118 of the neck strap 16, for example by high frequency welding. Fixing the side regions 66, 68 to the neck strap 16 in this way prevents the side regions 66, 68 being pulled back through the slits 112 when the neck brace 10 is in place, thereby securing the neck strap 16 in place.

Alternatively, the neck strap 16 may be releasably attachable from the frame 12 by means of fixings, to allow for cleaning or replacement.

It will be appreciated that many variations in the design of the instrument are possible within the scope of the invention. For example, the frame 12 need not be formed from a single continuous wire, but instead could be formed from multiple wires that are joined together. The brace section 20 and support section 18 need not be joined by side members 22, but may instead be shaped so as to extend directly into one another.

The neck strap 16 need not comprise overlapping closure sections 70, but may be fastened around the user's neck by any suitable means. For example, the neck strap 16 may be removably attachable to one of the side members 22, or the neck strap 16 may be formed from a continuous piece that is elasticated to allow the user to bring the neck brace 10 over the head.

1. A neck brace comprising:
a frame including a support section for supporting a user's lower jaw and a brace section for bracing the support section; wherein the brace section comprises a sternum rest and a strut that joins the sternum rest to the remainder of the frame and is shaped to avoid contact with the user's clavicle region.

2. The neck brace of claim 1, wherein the strut is a bridge member shaped to provide clearance over the user's clavicle region.

3. The neck brace of claim 2, wherein the bridge member is curved about an axis of curvature that, in use, is substantially parallel to a line extending across the user's clavicles.

4. (canceled)

5. The neck brace of claim 1, wherein the strut comprises downwardly-converging arms that taper from the remainder of the frame toward the sternum rest.

6. The neck brace of claim 5, wherein the strut is V-shaped when viewed from the user's front in use, with the sternum rest being located at a downward apex of the V.

7. (canceled)

8. The neck brace of claim 16, wherein an apex region of the strut is U-shaped and is defined by a pair of substantially parallel side portions spaced apart by at least one cross-member.

9. (canceled)

10. The neck brace of claim 1, wherein at least part of the strut is formed from an elongate wire.

11. The neck brace of claim 10, wherein the sternum rest comprises a pad removably attachable to the strut; and wherein the sternum pad has a peripheral formation shaped to engage the wire.

12. The neck brace of claim 11, wherein the peripheral formation is a groove extending at least partially around the sternum pad.

13. The neck brace of claim 11, wherein the wire exerts resilient engaging force on the sternum pad.

14. (canceled)

15. (canceled)

16. The neck brace of claim 1, wherein the sternum pad comprises a TENS unit.

17. (canceled)

18. The neck brace of claim 1, wherein the brace section and the support section are formed from a continuous elongate wire.

19. The neck brace of claim 18, wherein the support section has a jaw rest with a flattened, elongate cross-section in a plane orthogonal to the wire of the support section.

20. (canceled)

21. The neck brace of claim 1, wherein the neck brace further comprises a retainer for extending around the back of a user's neck to hold the support section and the brace section in a use position; and wherein the retainer comprises a TENS electrode.

22. (canceled)

23. A neck brace comprising at least one TENS electrode and having a holder for a TENS unit for providing pulses to the TENS electrode.

24. The neck brace of claim 23, wherein the TENS unit is removable from the holder and disconnectable from the TENS electrode.

25. The neck brace of claim 23 and comprising a support section for supporting a user's lower jaw, a brace section for bracing the support section and a retainer for extending around the back of a user's neck to hold the support section and the brace section in a use position, wherein the TENS electrode is on the retainer and the holder for the TENS unit is on the brace section.

26. (canceled)

27. The neck brace of claim 25, wherein the brace section is electrically conducting and the TENS electrode and a TENS unit in the holder are electrically connected via the brace section.

28. The neck brace of claim 27, wherein the brace section defines a gap such that the TENS unit, when located in the holder and activated, bridges the gap to complete an electrical circuit including the TENS electrode.

29. The neck brace of claim 25, wherein the brace section comprises a strut that extends downwardly to the holder for the TENS unit, positioned to lie adjacent the sternum of a user; and wherein the strut comprises a pair of arms that define the holder between them.

30. (canceled)

31. (canceled)

32. In combination, a neck brace as defined in claim 23 and a TENS unit cooperable with or fitted to the holder.

33. (canceled)

34. (canceled)
